(19) United States
(12) Patent Application Publication
(10) Pub. No.: US 2012/0225819 A1
(43) Pub. Date: Sep. 6, 2012

(54) STABLE FORMULATION OF FACTOR VIII

(76) Inventors: Jan Jezek, Wellingborough (GB); Barry Kingston Derham, Cambridge (GB)

(21) Appl. No.: 13/409,384

(22) Filed: Mar. 1, 2012

Related U.S. Application Data
(63) Continuation of application No. PCT/GB2010/051441, filed on Sep. 2, 2010.
(60) Provisional application No. 61/239,934, filed on Sep. 4, 2009.

(30) Foreign Application Priority Data
Sep. 4, 2009 (GB) 0915480.8

(51) Int. Cl. A61K 38/37; A61P 7/04

(52) U.S. Cl. 514/14.1

(57) ABSTRACT

The invention relates inter alia to a composition which is storage-stable at 25° C. for a minimum of 18 weeks comprising a therapeutically effective amount of Factor VIII and an aqueous medium having a Factor VIII potency after 18 weeks of storage at 25° C. of at least 90% of the Factor VIII potency of a Control Composition.

### RELATED APPLICATIONS

This application is a continuation of International Application No. PCT/GB2010/051441, which designated the United States and was filed on Sep. 2, 2010, published in English, which claims the benefit of U.S. Provisional Application No. 61/239,934, filed on Sep. 4, 2009. This application claims priority under 35 U.S.C. §119 or 365 to United Kingdom Application No. 0915480.8, filed Sep. 4, 2009. The entire teachings of the above applications are incorporated herein by reference.

### FIELD OF THE INVENTION

This invention relates to the stabilisation of coagulation Factor VIII, particularly in aqueous liquid compositions for therapeutic applications.

### BACKGROUND TO THE INVENTION

Haemophilia A is a hereditary disorder in which the clotting ability of blood is impaired and excessive bleeding results. Haemophilia A (often called classic haemophilia) is a deficiency in clotting factor VIII. Prolonged bleeding is the hallmark of haemophilia A. Small wounds and punctures are not usually a problem, but uncontrolled internal bleeding can result in pain, swelling and permanent damage especially to joints and muscles. Severity of symptoms can vary and severe forms become apparent early on. Mild cases may go unnoticed until later in life when there is excessive bleeding and clotting problems in response to surgery or trauma. Internal bleeding may happen anywhere, and bleeding into joints is common.

Factor VIII is a multi-domain glycoprotein which is essential to the blood clotting cascade and is used for the treatment of haemophilia A. Apart from treating bleeding episodes it is increasingly administered prophylactically to reduce long term damage to joints. Factor VIII is one of many proteins involved in the blood clotting cascade. Factor VIII is a cofactor for Factor IXa which, in the presence of calcium ions and phospholipids, converts factor X to the activated form Xa. Factor VIII molecule consists of six key domains denoted A1, A2, A3, B, C1 and C2. Most currently marketed Factor VIII products comprise all domains. However, the B-domain was shown dispensable for the coagulation activity, and one of the currently marketed Factor VIII products (ReFacto) is produced with the B-domain deleted. The native structure of Factor VIII comprises complexed calcium ions with an assumed 1:1 stoichiometry (i.e. one calcium ion per molecule of Factor VIII). Appropriate binding of calcium ions within the structure of Factor VIII is thus important for maintaining its structural integrity and coagulation activity.

All marketed Factor VIII products are currently presented as a lyophilized formulation of Factor VIII either produced by recombinant technology or purified from pooled plasma. The preparations are typically stabilized by addition of sucrose. Administration of the lyophilized product is a very complex procedure involving a number of steps to reconstitute the product and maintain the sterility of the formulation. See, for example, Prescribing Information for ADVATE, March 2010. Once reconstituted, shelf-life is limited to 3 hours. Dosage varies considerably depending on patient and situation (e.g. bleeding episodes). Administration by bolus infusion takes up to 5 minutes, at a maximum infusion rate of 10 mL per min. This administration is initially conducted in haemophilia centres, but once patients are capable of self-administration, and where practical considerations allow, the patient will self-administer at home.

However, a need exists to provide an easy-to-use formulation of Factor VIII which does not rely upon reconstitution. A stable aqueous formulation of Factor VIII would allow the development of a convenient patient-ready pre-filled syringe or containment in a conventional storage vial, replacing the current powder formulations.

Factor VIII is a relatively unstable protein, particularly in aqueous solutions. Stabilisation during manufacturing and storage by complexing with other plasma proteins, particularly von Willebrand factor (vWF) and albumin, has been described. See, for example, U.S. Pat. No. 6,228,613. The vWF:Factor VIII complex is not marketed as a therapeutic product but has been used in some commercial processes as a stable intermediate. Various albumin-free lyophilised formulations have also been disclosed. For example, U.S. Pat. No. 6,586,573 describes the use of stabilisers selected from the group consisting of sucrose, trehalose, raffinose and arginine and a bulking agent selected from the group consisting of mannitol, glycine and alanine. U.S. Pat. No. 5,919,766 discloses the use of a non-ionic surfactant in combination with L-histidine buffer and sodium or potassium chloride for protecting Factor VIII in the amorphous state. U.S. Pat. No. 5,565,427 discloses a stabilised formulation of Factor VIII comprising an amino acid or one of its salts or homologues and a detergent or an organic polymer such as polyethyleneglycol.

U.S. Pat. No. 5,605,884 discloses stabilised formulations of Factor VIII in high ionic strength media based on histidine buffer in the presence of calcium chloride and a high concentration of sodium chloride or potassium chloride. Such compositions were shown to improve significantly the stability of Factor VIII in aqueous form following reconstitution.

The importance of calcium ions in the formulations of Factor VIII is generally recognised (see Fatourus et al (1997) Int J Pharm 155, 121-131). All Factor VIII products currently available in the market contain calcium chloride, at a concentration between 2-10 mM following reconstitution. According to U.S. Pat. No. 6,599,724, the presence of other divalent cations, namely Cu²⁺ and Zn²⁺, optionally in the presence of Ca²⁺ ions or Mn²⁺ ions improves the stability of Factor VIII.

The addition of EDTA to Factor VIII formulations to reduce degradation by metal dependent proteases is described in WO96/15150.

Thus, while the prior art suggests that aqueous stable Factor VIII formulations exist, none provide for aqueous formulations appropriate for long term storage.

### SUMMARY OF THE INVENTION

The invention relates to the discovery of a storage stable aqueous composition of Factor VIII. The composition is storage-stable at 25° C. for a minimum of 18 weeks comprising a therapeutically effective amount of Factor VIII and an aqueous medium having a Factor VIII potency after 18 weeks of storage at 25° C. of at least 90%, preferably at least 95%, of the Factor VIII potency of a Control Composition. Alternatively or additionally, the composition is storage-stable at 5° C. for a minimum of 26 weeks, such as 52 weeks, comprising a therapeutically effective amount of Factor VIII and an aqueous medium having a Factor VIII potency after 26 or 52 weeks of storage at 5° C. of at least 90%, preferably at least 95% and more preferably at least 98%, of the Factor VIII potency of a Control Composition.

For example, as an aspect of the invention, there is a provided a composition which is storage-stable at 25° C. for a minimum of 18 weeks and at 5° C. for a minimum of 26 weeks comprising a therapeutically effective amount of Factor VIII and an aqueous medium having a Factor VIII potency after 18 weeks of storage at 25° C. and after 26 weeks of storage at 5° C. of at least 95% of the Factor VIII potency of a Control Composition.

It is particularly beneficial to provide the formulation with an excess of Ca²⁺ ions in the presence of a strong ligand, such as EDTA. The strong ligand desirably binds undesired transition metal ions which may adversely impact on the Factor VIII potency in the formulation. In this context, “an excess” means that there are present free Ca²⁺ ions which are not either complexed to Factor VIII or to the strong ligand (or any other ligand).

The invention further relates to lyophilizates, dried e.g. spray-dried or other substantially water-free compositions corresponding to the compositions described herein. Such dried compositions can be suitable for reconstitution and can be packaged in a sterile, sealed vial or container together with water for injection. Such reconstituted products will have advantages of extended shelf life over other Factor VIII formulations currently commercially available. Thus the invention provides a substantially water-free composition obtainable by removing water, such as by lyophilization or by drying e.g. spray-drying from a composition according to the invention.

### DETAILED DESCRIPTION OF THE INVENTION

The present invention relates to the discovery of stable aqueous compositions of Factor VIII. In order to make an aqueous composition suitable for therapeutic application, such as intravenous, subcutaneous or intramuscular application, certain desirable characteristics of the composition must be ensured, such as safety and regulatory acceptance of the excipients. The key aqueous compositions of Factor VIII disclosed herein are based on excipients already approved by regulatory authorities as inactive ingredients in drug products for parenteral applications.

Factor VIII potency is defined as the value measured using a CA-50 semi-automated coagulometer (Sysmex Corporation) and the APTT procedure provided by Dade Behring Inc. (OTXW G13 E0535 (623) H1, April 2001 edition) for determination of coagulation Factor VIII. It will be understood that where the assay or coagulometer are unavailable or inconvenient, equivalent assays can be substituted therefor, using the teachings of the present application as guidance.

“Control Composition” is defined herein as a composition possessing the same components and excipients in the same concentrations without being subjected to the storage conditions. The Control Compositions can be prepared fresh (e.g., from lyophilized or recently made/isolated Factor VIII) or can be measured for potency prior to the composition being subjected to the storage conditions. Control Compositions do not include Factor VIII compositions that have been reconstituted or stored for more than 3 hours under any conditions or under conditions that cause substantial reduction in Factor VIII potency.

It is also understood that storage for substantial periods of time is expected to be subjected to varied conditions. Typical storage at 25° C. will actually include changes or variations in temperature typical for room temperature storage. Typically, where the composition is subjected to a controlled stability test, the temperature will be maintained within 3° C. of the stated temperature. However, it is understood that the testing can also be conducted with a product obtained, for example, from a commercial lot at the point of sale or at the time of administration. In these instances, it is understood that the storage temperatures, particularly room temperature storage, may not be as tightly controlled and may vary by 10° C. or more. Such variations in storage conditions are intended to be included within the scope of the claims.

Preferably the composition comprises water, a therapeutically effective amount of Factor VIII, TRIS, benzoate, calcium ion, EDTA, and an alkali metal ion, such as sodium or potassium and, optionally a surfactant and/or a preservative. In preferred embodiments, the composition consists essentially of these components. A composition consisting essentially of the stated components is intended to exclude compositions that contain excipients or additives that result in the reduction of Factor VIII potency under the conditions of storage. For example, a composition consisting essentially of the stated components is intended to exclude a composition which contains an excipient having a pKa within 1 pH unit of the pH of the formulation and/or a strong ligand in amounts which exceed the concentration of free metal ions present in the formulation. The pH is preferably about 6.25.

The Factor VIII is preferably present in the composition in an amount between 50 and 1000 IU/ml, preferably between 50 and 250 IU/ml. IU is understood to mean international units, as defined by the WHO. The invention is applicable to recombinant Factor VIII as well as Factor VIII purified from pooled plasma. The terms “coagulation Factor VIII” and “Factor VIII” are used herein to encompass a protein molecule, either produced by recombinant technology or purified from pooled plasma with biological activity identical or similar to that of the native human Factor VIII. The terms “coagulation factor VIII” and “Factor VIII” encompass both molecules containing all native domains of Factor VIII (A1, A2, A3, B, C1 and C2) and molecules in which one or more domains have been deleted without significantly affecting the blood clotting activity, for example, with the B-domain deleted. The terms “coagulation Factor VIII” and “Factor VIII” encompass both molecules comprising domains with amino acid sequence identical to the native human Factor VIII as well as analogues in which mutations of the amino acid sequence have been implemented without significantly affecting the coagulation activity.

The Factor VIII composition preferably contains a sufficient concentration of calcium ions to optimize Factor VIII potency and preferably is present in the composition in excess to that required for complexation with protein and more particularly with protein and any strong ligands in the composition. Accordingly, calcium is preferably added in an amount and in a suitable form to provide calcium ions (free and complexed) at a concentration between 0.5 to 30 mM, preferably between 2 to 20 mM, most preferably between 5 mM to 15 mM. The calcium can be added to the composition, for example as a salt. A preferred example of a calcium salt includes calcium chloride. As will be explained below, calcium carbonate and calcium hydrogen carbonate are preferably excluded. Optionally, magnesium ion, such as magnesium chloride, can be added. In another embodiment, magnesium can be excluded.

The composition further preferably contains a strong ligand in an amount sufficiently low to allow the presence of free calcium ions in the composition. The term “ligand” is used herein to encompass any compound capable of binding metal ions resulting in formation of complex ions. For the purpose of this invention the ligands are divided to “weak ligands”, “medium-strength ligands” and “strong ligands”. The terms of “weak ligand”, “medium-strength ligand” and “strong ligand” are defined based on the stability constants of their complexes with calcium ion, as follows: A weak ligand has a stability constant of a complex with calcium ion log K<0.5; a medium-strength ligand has stability constant of a complex with calcium ion log K between 0.5 to 2, a strong ligand has stability constant of a complex with calcium ion log K>2. All stability constants are those measured at 25° C. A strong ligand is preferably added to the composition to control or minimize undesirable protein-metal ion complexation. Thus, the preferred amount of ligand to be added is that which binds undesirable metal ions (e.g., residual or trace transition metals, such as ions of copper, zinc or iron or manganese). The strong ligand desirably has a binding affinity for transition metal ions such as Cu²⁺, Zn²⁺, Fe³⁺) or Mn²⁺ which exceeds its binding affinity for Ca²⁺ ions. Binding affinity is suitably measured in terms of the stability constant of a complex of the strong ligand with said ion measured at 25° C. For example, the stability constants of EDTA complexes with Ca²⁺, Cu²⁺, Zn²⁺, Fe³⁺ ions are respectively 10.7, 16.5, 18.8 and 25.7 at 25° C. However, the preferred amount of ligand is preferably not so great as to compete and prevent desirable calcium ion complexation to the Factor VIII protein nor to bind all calcium ions in the composition. The presence of the strong ligand will thus substantially eliminate the presence of metal ions other than calcium without affecting the desirable level of free calcium ion in the composition. This preferred range of ligand is defined herein as an “effective amount.” The stability constants of metal-ligand complexes can be obtained from a comprehensive database published by the US National Institute of Standards and Technology (NIST Standard Reference Database 46, R. M. Smith and A. E. Martell: Critically Selected Stability Constants of Metal Complexes Database). The art of using the stability constants in the context of the present invention is described in detail in WO2009/133200 in the name of Jan Jezek, which is incorporated herein by reference. The term “free calcium ions” is used herein to denote calcium ions that are not bound by Factor VIII or by a strong ligand (or any other ligand). For the purposes of this definition, water (which may solvate the calcium ions) is not considered to be a ligand.

Examples of suitable strong ligands (calcium ion Log K's are in parentheses) include: EDTA (10.81), citrate (3.48), methionine (2.04), cysteine (2.5), malate (2.06), and sulphite (2.62). The selection of ligands is described generally in WO2009/133200, which is incorporated herein by reference. For the purpose of binding undesirable metal ions, use of a strong ligand is preferred and most preferably the strong ligand is EDTA.

Thus, preferably, EDTA is present at a concentration allowing the presence of free calcium ions in the composition. For example, a preferred composition comprises EDTA at a concentration between 0.001 mM to 2 mM.

The pH of the composition is preferably about 6.25. At this pH it is considered by the Inventors that Factor VIII is most stable. WO2008/084237 describes methods of controlling the pH of an aqueous composition utilizing displaced buffers. The publication is incorporated herein by reference in its entirety. Displaced buffers are buffers having ionisable groups having a pKa within 1-5 e.g. 1-4 e.g. 1-3 pH units of the pH of the composition and having no pKa values within 1 pH unit of the pH of the composition. Suitable displaced buffer combinations include one displaced buffer having an ionisable group with a pKa above the pH of the composition and one displaced buffer with an ionisable group with a pKa below the pH of the composition. Alternatively a displaced buffer may contain two ionisable groups, one with a pKa above the pH of the composition and one with an ionisable group with a pKa below the pH of the composition. Preferred buffers can be selected in accordance with the teachings of that reference. Particularly preferred displaced buffers include TRIS and benzoate which may be used in combination and especially in a composition of pH between 5.5 and 7 e.g. about 6.25. Preferably, the TRIS and benzoate are present each at a concentration of between 1 to 100 mM, preferably between 5 to 50 mM, most preferably between 10 to 30 mM.

In order to avoid inconvenient complexation of desirable calcium ions, the buffers employed should themselves be weak ligands.

Preferably, the composition additionally contains sodium or potassium ions to improve the ionic strength of the composition. These ions can be conveniently added as a salt, such as sodium chloride or potassium chloride. Thus, a preferred composition comprises at least 100 mM of sodium chloride or potassium chloride. Alternative salts such as sodium sulfate or potassium sulfate can also be considered.

Surfactants can also be optionally added to the composition. Preferred surfactants include polysorbate 20, polysorbate 60, polysorbate 80, poloxamer 188 or poloxamer 407. The surfactants can preferably be added in an amount up 10 mg/ml, such as up to 5 mg/ml, such as 3 mg/ml. Preferably, the composition comprises polysorbate 80 at a concentration between 10 to 50 mg/L or poloxamer 188 at a concentration between 0.2 to 3 mg/mL.

The composition can also optionally comprise a preservative, such as those approved for use in drug products. Preferred preservatives can be selected from the group comprising phenol, m-cresol, benzylalcohol, propylparaben, benzalkonium chloride and benzethonium chloride.

In embodiments, 1,2-propanediol can be added, such as at a concentration of at least 100 mM. Preferably, the above composition comprises 1,2-propanediol at a concentration between 100 mM to 1 M, most preferably between 200 mM to 500 mM.

It has been discovered that controlling the headspace gas within the sealed vial can improve stability as well. In particular, it has been discovered that carbon dioxide is detrimental to Factor VIII stability. Thus, the invention relates to compositions stored in a sealed container under a headspace substantially free of carbon dioxide. For example, nitrogen or helium can be conveniently used to fill the head space of the sealed container.

A composition for injection must be sterile. Sterility of a liquid composition for therapeutic use can be achieved by filtering the composition prior to the final filling to an appropriate container, such as a vial or a pre-filled syringe, under sterile conditions, using an appropriate filter or membrane, such as a 0.22 μm filter or a 0.45 μm filter. The preferred aqueous compositions of Factor VIII disclosed herein are sterile-filtered and filled aseptically into the final container. The compositions can also be used in the presence of a pharmacologically acceptable preservative.

The present invention discloses an aqueous formulation of Factor VIII in which the potency of Factor VIII is preserved for extended period of time both at 5° C. and at 25° C. An important aspect of the present invention lies in controlling the metal ions, e.g., adding calcium ions and avoiding excess or free forms of medium-strength and strong ligands, thus ensuring the presence of free calcium ions in the solution. As used herein, “free forms” of ligands are forms of ligands which are not complexed to a metal ion. Buffers, e.g., displaced buffers, are preferably selected among weak ligands in relation to calcium ion binding.

It is important to realize in the context of the present invention that molecules of dissolved gases are also ligands capable of forming co-ordinate bonds with metal ions. This is particularly the case of carbon dioxide. Carbon dioxide-metal complexes are reviewed in Gibson D. H.: Coordination Chemistry Reviews, 185-186 (1999) 335-355. Apart from direct binding to a metal ion, carbon dioxide can also contribute to metal binding indirectly by giving rise to various carbonate species that are capable of metal-binding. This is due to the fact that in aqueous solutions carbon dioxide always exists in equilibrium with carbonic acid and various carbonate anions:

CO₂+H₂O [symbol] H₂CO₃ [symbol] H⁺HCO₃⁻ [symbol] 2H⁺CO₃²⁻

Experiments showed that the presence of carbon dioxide in the headspace, even at a low partial pressure, is detrimental to the stability of aqueous Factor VIII stored under the headspace. This is the reason why use of calcium as calcium carbonate or calcium hydrogen carbonate is suitably avoided.

The optimum pH for shelf stability of Factor VIII is about 6.25. Furthermore, the stability of Factor VIII was found experimentally to be improved in the presence of ionic species. This is in line with previous reports (e.g. U.S. Pat. No. 5,605,884). However, it is important to ensure that the ionic species do not comprise free forms of medium-strength or strong ligands in relation to calcium ion binding. The preferred ionic species in the context of the present invention are sodium or potassium cations and chloride anions. Compositions of Factor VIII according to all aspects of the present invention preferably comprise at least 100 mM sodium chloride or at least 100 mM potassium chloride.

Compositions of the invention are suitably free of heparin or a heparin salt such as heparin sodium.

Compositions of the invention are suitably free of arginine.

Compositions of the invention are suitably free of glycine.

In one aspect of the present invention, an aqueous composition comprises a therapeutically relevant concentration, or effective amount, of Factor VIII, further characterized in that:
(i) the composition comprises calcium ions at concentration between 0.5 to 30 mM, preferably between 2 to 20 mM, most preferably between 5 mM to 15 mM;
(ii) the composition is substantially free of excipients which are free forms of medium-strength ligands or strong ligands;
(iii) the pH of the composition is adjusted to about 6.25.

Surprisingly, it was found beneficial to add to such compositions a small amount of a strong ligand, such as EDTA. It is critical, however, that the concentration of the strong ligand does not exceed the concentration of calcium ion present in the composition. Preferably the concentration of the strong ligand is less than half of the concentration of calcium ion, for example one tenth of the concentration of the calcium ion. The strong ligand is then practically absent in its free (i.e. not bound to metal ion) form. It is believed that the simultaneous presence of calcium ion and the strong ligand has the benefit of removing traces of other metal ions (such as transition metal ions e.g. cupric or ferric ions or other ions of copper, iron, zinc and manganese) which may otherwise be present in the composition as contaminants and contribute to detrimental oxidation or aggregation processes. As noted above, the strong ligand desirably has a binding affinity for transition metal ions such as Cu²⁺, Zn²⁺, Fe³⁺) or Mn²⁺ which exceeds its binding affinity for Ca²⁺ ions.

Therefore, in another aspect of the present invention, an aqueous composition comprises a therapeutically relevant concentration, or effective amount, of Factor VIII, further characterized in that:
(i) the composition comprises calcium ions at concentration between 0.5 to 30 mM, preferably between 2 to 20 mM, most preferably between 5 mM to 15 mM;
(ii) the pH of the composition is adjusted to about 6.25;
(iii) the composition comprises a strong ligand at a concentration no higher than that of calcium ions; the preferred strong ligand is EDTA;
(iv) the composition is substantially free of other excipients which are free forms of medium-strength ligands or strong ligands.

Preferred compositions according to the present invention comprise a buffer system based on a combination of benzoate ion and tromethamine (TRIS). Such buffering system is in accord with disclosures made in WO2008/084237 as it is based on components which have pKₐ values at least 1 unit more or less than the pH of the composition at the intended temperature range of storage of the composition. In another aspect of the present invention, an aqueous composition comprises a therapeutically relevant concentration, or effective amount of Factor VIII, further characterized in that:
(i) the composition comprises calcium ions at concentration between 0.5 to 30 mM, preferably between 2 to 20 mM, most preferably between 5 mM to 15 mM;
(ii) the composition comprises benzoate ion and TRIS, each at concentration between 1 to 100 mM, preferably between 5 to 50 mM, most preferably between 10 to 30 mM;
(iii) the composition is substantially free of excipients which are free forms of medium-strength ligands or strong ligands;
(iv) the pH of the composition is adjusted to about 6.25.

In yet another aspect of the present invention, an aqueous composition comprises a therapeutically relevant concentration, or effective amount, of Factor VIII, further characterized in that:
(i) the composition comprises calcium ions at concentration between 0.5 to 30 mM, preferably between 2 to 20 mM, most preferably between 5 mM to 15 mM;
(ii) the composition comprises benzoate ion and TRIS, each at concentration between 1 to 100 mM, preferably between 5 to 50 mM, most preferably between 10 to 30 mM;
(iii) the pH of the composition is adjusted to about 6.25;
(iv) the composition comprises a strong ligand at a concentration no higher than that of calcium ions; the preferred strong ligand is EDTA;
(v) the composition is substantially free of other excipients which are free forms of medium-strength ligands or strong ligands.

In yet another aspect of the present invention, lyophilizates, spray-dried or other substantially water-free compositions are obtainable by removing water from the aqueous compositions described herein. For this purpose, the compositions can also comprise one or more additional excipients serving as cryoprotectants, vitrification agents and/or bulking agents. Preferred additional excipients are selected from sucrose, trehalose, lactose, raffinose, glycerol, mannitol, xylitol and sorbitol. Such dried compositions can be suitable for reconstitution and can be packaged in a sterile, sealed vial or container together with water or other reconstitution medium required for injection. Such dry and reconstituted products may be expected to have advantages of extended shelf life over other Factor VIII formulations currently commercially available.

Compositions according to all aspects of the present invention have preferably one or more of the following features:
(i) the composition is sterile and filled aseptically into a suitable container such as a sterile vial, ampoule or pre-filled syringe; the sterility can be achieved by filtering the composition prior to the final filling to the container using an appropriate filter or membrane, such as a 0.22 μm filter or a 0.45 μm filter; the composition may also contain a pharmaceutically acceptable preservative, such as phenol, m-cresol or benzylalcohol;
(ii) the composition comprises a pharmaceutically acceptable surfactant, such as polysorbate 20, polysorbate 40, polysorbate 60, polysorbate 80, poloxamer 188 or poloxamer 407;
(iii) the composition comprises 1,2-propanediol at a concentration between 100 mM to 1 M, most preferably between 200 mM to 500 mM;
(iv) the composition is stored under a headspace substantially free from carbon dioxide, such as nitrogen or helium headspace; alternatively the composition is stored in a sealed container with no headspace.

Another aspect of the present invention describes an optimized aqueous composition of Factor VIII which comprises a therapeutically relevant concentration, or effective amount, of Factor VIII, further characterized in that:
(i) the composition comprises calcium ions at concentration between 8 to 15 mM;
(ii) the composition comprises benzoate ion and TRIS, each at concentration between 10 to 25 mM e.g. 15 to 25 mM;
(iii) the composition comprises sodium chloride at concentration between 200 to 500 mM e.g. 300 to 500 mM;
(iv) the pH of the composition is adjusted to about 6.25;
(v) the composition comprises EDTA at a concentration between 0.25 mM to 0.5 mM;
(vi) the composition comprises polysorbate 80 at a concentration between 10 to 50 mg/L or poloxamer 188 at a concentration between 0.2 to 3 g/L e.g. 0.5 to 3 g/L;
(vii) the composition is stored in a sealed container under a headspace substantially free from carbon dioxide, such as nitrogen or helium headspace;
(viii) the composition is sterile.

For example, an aspect of the present invention describes an optimized aqueous composition of Factor VIII which comprises a therapeutically relevant concentration, or effective amount, of Factor VIII, further characterized in that:
(i) the composition comprises calcium ions at concentration between 8 to 15 mM;
(ii) the composition comprises benzoate ion and TRIS, each at concentration between 10 to 25 mM;
(iii) the composition comprises sodium chloride at concentration between 200 to 500 mM;
(iv) the pH of the composition is adjusted to about 6.25;
(v) the composition comprises EDTA at a concentration between 0.25 mM to 0.5 mM;
(vi) the composition comprises polysorbate 80 at a concentration between 10 to 50 mg/L or poloxamer 188 at a concentration between 0.5 to 3 g/L;
(vii) the composition is stored in a sealed container under a headspace substantially free from carbon dioxide, such as nitrogen or helium headspace;
(viii) the composition is sterile.

A particularly preferred aqueous composition of the invention comprises:
(i) a therapeutically effective amount of Factor VIII;
(ii) calcium ions at concentration between 8 to 15 mM;
(iii) benzoate ion and TRIS, each at concentration between 15 to 25 mM;
(iv) sodium chloride at concentration between 300 to 500 mM;
(v) EDTA at a concentration between 0.25 mM to 0.5 mM;
(vi) polysorbate 80 at a concentration between 10 to 50 mg/L or poloxamer 188 at a concentration between 0.2 to 3 mg/mL e.g. 0.5 to 3 mg/mL;
   wherein the composition has a pH adjusted to about 6.25 and is stored in a sealed container under a headspace substantially free from carbon dioxide, such as nitrogen or helium headspace.

The potency of Factor VIII can be estimated in vitro by measuring the coagulation time in the activated partial thromboplastin time (APTT) test or by a specific Factor VIII chromogenic assay as described herein. Such methods are accepted by the US and the European Pharmacopoeia.

Benzoic acid or its salts, such as sodium or potassium salt, can be used as the source of benzoate anion in the context of the present invention. Either TRIS base or TRIS hydrochloride can be used as a source of TRIS. Calcium chloride is the preferred source of calcium ions, but other soluble salts of calcium can also be used.

The term “therapeutically relevant concentration of Factor VIII” is used herein to describe a concentration of Factor VIII in an aqueous composition which is used in therapy on human or animal body. This includes well established routes of administration, such as intravenous infusion, as well as novel routes of administration which may be enabled by the current invention, such as pump administration. The therapeutically relevant concentration of Factor VIII in currently marketed products following reconstitution is typically between 100 to 300 IU/mL. However, the present invention is also applicable to higher concentrations, such as higher than 500 IU/mL or higher than 100 IU/mL.

The term “free form of a ligand” is used herein to describe molecules of a ligand which is not bound to a metal cation in a particular composition comprising ligand molecules and metal ion molecules. One of ordinary skill in the art will be able to calculate the proportion of free ligand from stability constants of the ligand-metal-ion complex providing overall concentrations of all ligands and all metal ions in the composition are known.

One skilled in the art will appreciate that in the context of the present invention a pH “around” or “about” 6.25 means a pH range within which the rates of major degradation processes are not considerably different from those measurable at pH 6.25, preferably a pH range between 5.9 to 6.6, most preferably 6.1 to 6.4.

The invention is illustrated in the following examples:

### Example 1

### Preservation of Factor VIII Potency in Reconstituted Marketed Factor VIII Composition and in a Stabilized Formulation Measured by a Two-Step Clotting Assay

Measurements of Factor VIII potency were made using CA-50 semi-automated coagulometer (Sysmex Corporation). APTT procedure provided by Dade Behring Inc. (OTXW G13 E0535 (623) H1, April 2001 edition) for determination of coagulation Factor VIII was followed. Residual potencies were calculated relative to the activity measured in the samples at the start of the stability trial. All measurements were carried out in single measurements. The preservation of potency was compared between the reconstituted composition of a lyophilized marketed Factor VIII product (HELIXATE®, Bayer Schering Corp.) and a formulation of the same active material stabilized in accordance to the invention.

HELIXATE is said to contain the following components:

### (Helixate® FS Prescribing Information, August, 2009):

| | | |
|---|---|---|
| | 250 IU, 500 IU, | |
| Stabilizer | 1000 IU | 2000 IU, 3000 IU |

| | | |
|---|---|---|
| Sucrose | 0.9-1.3% | 0.9-1.2% |

| | | | | |
|---|---|---|---|---|
| Glycine | 21-25 | mg/mL | 20-24 | mg/mL |
| Histidine | 18-23 | mmol/L | 17-22 | mmol/L |

| | 250 IU, 500 IU, | | | |
|---|---|---|---|---|
| Inactive Ingredient/Excipient | 1000 IU | | 2000 IU, 3000 IU | |
| Sodium | 27-36 | mEq/L | 26-34 | mEq/L |
| Calcium | 2.0-3.0 | mmol/L | 1.9-2.9 | mmol/L |
| Chloride | 32-40 | mEq/L | 31-38 | mEq/L |
| Polysorbate 80 | 64-96 | μg/mL | 64-96 | μg/mL |
| Sucrose | 28 | mg/vial | 52 | mg/vial |

| | | |
|---|---|---|
| Imidazole, tri-n-butyl | Trace amounts | Trace amounts |
| phosphate, and copper | | |

Each vial of Helixate FS is said to contain the labeled amount of recombinant factor VIII in international units (IU). One IU, as defined by the World Health Organization standard for blood coagulation factor VIII, human, is approximately equal to the level of factor VIII activity found in 1 mL of fresh pooled human plasma.

The formulation stabilized in accordance to the invention contains:

| Excipients/Active | Concentration | |
|---|---|---|
| FVIII | 50 to 250 | IU/mL |
| TRIS | 21 | mM |
| Potassium benzoate | 21 | mM |
| Calcium chloride | 8 | mM |
| EDTA | 0.5 | mM |
| Tween 80 | 25 | mg/L |
| Sodium chloride | 500 | mM |

All formulations were prepared at 150 IU/mL factor VIII activity. The stabilized formulations were prepared by a three-step dialysis of the reconstituted HELIXATE® product against the new formulation and subsequent adjustment of volume to achieve the required specific activity of the Factor VIII. Residual potency measured following incubation at 4° C., 25° C. and 37° C. is shown in Table 1. A considerable improvement in the preservation of FVIII potency was observed in the stabilized sample compared with the reconstituted marketed formulation. Whilst the control formulation showed >50% loss of potency following two weeks of storage at both 37° C. and 25° C. and 26 weeks of storage at 4° C., the potency remained within 10% of the original value in the stabilized liquid composition following 52 weeks of storage at 4° C. and 13 weeks of storage at

25° C. The loss of potency in the stabilized composition was more rapid at 37° C., but >67% was still retained after 2 weeks and >42 after 6 weeks of storage, demonstrating considerably improved stability over that in reconstituted marketed composition of HELIXATE®.

TABLE 1
Preservation of Factor VIII potency following incubation
at 5 ± 3° C. 25 ± 2° C. 37 ±
2° C. for indicate period of time. Measured by
two-step clotting assay (Dade Behring)

| | Residual potency (%) | | | | | |
|---|---|---|---|---|---|---|
| Time/ | 5 ± 3° C. | | 25 ± 2° C. | | 37 ± 2° C. | |
| weeks | Control | Stabilized | Control | Stabilized | Control | Stabilized |
| 2 | | | 38.3% | 105.7% | 1.1% | 67.8% |
| 5 | 89.2% | 102.7% | | | | |
| 6 | | | 14.7% | 95.3% | 3.4% | 42.6% |
| 13 | | | 4.0% | 90.4% | | |
| 14 | | | | | | |
| 26 | 34.8% | 102.4% | | | | |
| 52 | 23.4% | 96.3% | | | | |

### Example 2

### Preservation of Factor VIII Potency in Stabilized Formulations

Measurements of Factor VIII potency were made using the Coatest SP4 Factor VIII chromogenic assay kit (Chromogenix, Instrumentation Laboratory Company, Lexington, USA) following the kit manufacturer's recommended method adapted for use on the Futura Analyser (Instrumentation Laboratory, Warrington, UK). All samples underwent an initial dilution to approximately 1 IU/mL in FactorVIII-deficient plasma (with normal VWF content) prior to further dilutions made using the kit buffer. All measurements were carried out in triplicates from which the mean value was calculated. Residual potencies were calculated relative to the activity measured in a frozen reference sample of identical composition. Residual potency was measured following incubation at 4° C., 25° C. and 37° C. in an aqueous composition of Factor VIII (151 IU/mL) adjusted to pH 6.25 comprising the following excipients: TRIS (21 mM), potassium benzoate (21 mM), sodium chloride (500 mM), calcium chloride (8 mM), EDTA (0.5 mM) and Tween 80 (25 mg/L). All compositions were stored under a nitrogen headspace. The liquid composition was prepared by a three-step dialysis of a reconstituted lyophilized product (KOGENATE®, Bayer Schering Corp.) against the new formulation ad subsequent adjustment of volume to achieve the required concentration.

KOGENATE® is said to contain the following components:

| Excipients/Active | Concentration | |
|---|---|---|
| rFVIII | 50 to 400 | IU/mL |
| Sodium Chloride | 1.76 | mg/mL |
| Calcium Chloride | 0.28 | mg/mL |
| Polysorbate 80 | 0.008 | mg/mL |
| Glycine | 23.2 | mg/mL |
| Histidine | 3.2 | mg/mL |
| Sucrose | 11.2 | mg/mL |

The residual potency of Factor VIII estimated in the sample stored at 4° C. was 105.2% following 18 weeks storage. The residual potency of Factor VIII estimated in the sample stored at 25° C. was 96.9% following 7 weeks storage and 96.2% following 18 weeks storage. The residual potency of Factor VIII estimated in the sample stored at

37° C. was 79.9% following 2 weeks incubation and 28.4% following 7 weeks storage. In addition, analysis of the results (1, 2, 4, 7, 10, 14 and 18 weeks storage at 4° C., 25° C. and 37° C.) using the Arrhenius equation allowed the generation of predicted rates of degradation from the observed loss of potency at the three temperatures. Mean predictions of loss relative to the frozen reference sample indicated the following predicted loss of potency per year: at 4° C. the mean prediction of loss per year was 0.475% (upper 95% confidence limit=4.142%). At 25° C. the mean prediction of loss per year was 21.789% (upper 95% confidence limit=19.554%).

### Example 3

### Effect of Headspace on the Preservation of Factor VIII Potency

The effect of headspace on the stability of Factor VIII in an aqueous solution was investigated by measuring the residual potency after 6 weeks of storage of a formulation containing Factor VIII, adjusted to pH 6.25 and comprising the following excipients: TRIS (21 mM), potassium benzoate (21 mM), sodium chloride (500 mM), calcium chloride (4 mM) and Tween 80 (25 mg/L). The liquid composition was prepared by a three-step dialysis of a reconstituted lyophilized product (KOGENATE®, Bayer Schering Corp.) against the new formulation and subsequent adjustment of volume to achieve the required concentration. The residual potency was expressed with respect to the potency measured in the sample prior to the storage trial. 100 microlitres of sample was kept in a 300 microlitre vial, flushed thoroughly with the specified gas and sealed. The effect of air, nitrogen, oxygen and carbon dioxide headspace was compared. The residual potency measured after the 6 weeks storage at 25° C. was 86.7% under the air headspace, 98.1% under the nitrogen headspace, 95.3% under the oxygen headspace and <1% under the carbon dioxide headspace. This experiment indicated clearly the detrimental effect of dissolved carbon dioxide on Factor VIII.

### Example 4

### Effect of the Presence of Calcium Ions (with and without EDTA) on the Activity of Factor VIII in Cell Culture Medium

The effect of calcium ions and EDTA was investigated on the Factor VIII activity measurable in expression media containing calls expressing Factor VIII. Baby hamster kidney (BHK) cells (adherent) transfected with Factor VIII gene were maintained in D-MEM/F-12 (Invitrogen) comprising 100 μg/mL geneticin, penicillin G and streptomycin. Cells were seeded in this medium and allowed to reach 70-80% confluence. For the Factor VIII production, the cells were transferred into a serum-free AIM-V Medium (Invitrogen). The AIM-V contained streptomycin sulfate at 50 μg/mL and gentamicin at 10 μg/mL. This basic composition was used as a control medium and compared with two other media of the same basic composition spiked with (i) 3 mM calcium chloride and (ii) 3 mM calcium chloride and 0.5 mM EDTA. Cells were allowed to express Factor VIII for 4 days. After this time the cells were removed and the activity of Factor VIII was followed in the medium for up to 20 days at ambient temperature. It was shown that the presence of the additional source of calcium or calcium/EDTA did not affect significantly the production of Factor VIII in the AIM-V Medium. However, it was shown that the presence of the additional source of calcium ions resulted in considerably higher preservation of Factor VIII activity in the medium compared with the control medium in the absence of the additional calcium ion (3 mM). Importantly, the preservation of Factor VIII was even more considerable in the medium spiked both with 3 mM calcium chloride and 0.5 mM EDTA (Table 1). Such stabilisation of Factor VIII in the cell culture medium could potentially improve the yield of recombinant factor VIII production considerably.

TABLE 2
Effect of CaCl₂ on the preservation of Factor VIII activity
expressed by transfected BHK cells in AIM-V media.

| | Factor VIII activity (IU/mL) | | |
|---|---|---|---|
| | | | AIM-V + 3 mM |
| | | AIM-V + 3 mM | CaCl₂ + 0.5 mM |
| Time | AIM-V | CaCl₂ | EDTA |
| Factor VIII expression | | | |
| phase | | | |
| 0 days | 0 | 0 | 0 |
| 1 day | 0.6 | 0.7 | 1.1 |
| 2 days | 1.5 | 1.2 | 1.1 |
| 3 days | 1.6 | 1.5 | 1.5 |
| 4 days | 1.8 | 1.7 | 1.7 |
| Subsequent phase | | | |
| (following removal of | | | |
| cells) | | | |
| 5 days | 1.7 | 1.7 | 1.8 |
| 7 days | 1.2 | 1.8 | 1.9 |
| 9 days | 0.8 | 1.4 | 2.0 |
| 12 days | 0.4 | 1.2 | 1.9 |
| 20 days | 0.3 | 1.1 | 1.9 |

As can be seen from Table 2, the addition of calcium ions (with or without strong ligand EDTA) did not have a significant effect on the Factor VIII activity during the expression phase. However, the activity declined less rapidly in the subsequent phase, following removal of the cells, in the presence of added calcium ions suggesting that the addition of calcium ions improves the stability of Factor VIII in the culture medium. The effect of adding the strong ligand EDTA is to increase the stability yet further.

While this invention has been particularly shown and described with references to preferred embodiments thereof, it will be understood by those skilled in the art that various changes in form and details may be made therein without departing from the scope of the invention encompassed by the appended claims.

Throughout this specification and claims, the word “comprise” or variations such as “comprises” or “comprising” will be understood to imply the inclusion of a stated integer or group of integers but not the exclusion of any other integer or group of integers.

The invention embraces all combinations of preferred and more preferred groups and embodiments of groups recited above.

1. A composition which is storage-stable at 25° C. for a minimum of 18 weeks comprising a therapeutically effective amount of Factor VIII and an aqueous medium having a Factor VIII potency after 18 weeks of storage at 25° C. of at least 90% of the Factor VIII potency of a Control Composition.

2. A composition which is storage-stable at 25° C. for a minimum of 18 weeks and at 5° C. for a minimum of 26 weeks comprising a therapeutically effective amount of Factor VIII and an aqueous medium having a Factor VIII potency after 18 weeks of storage at 25° C. and after 26 weeks of storage at 5° C. of at least 95% of the Factor VIII potency of a Control Composition.

3. A composition according to claim 1 comprising water, a therapeutically effective amount of Factor VIII, TRIS, benzoate, calcium ion, EDTA, and an alkali metal ion.

4. A composition according to claim 3, wherein the pH is about 6.25.

5. The composition according to claim 3, wherein the calcium ions are present at a concentration between 0.5 to 30 mM, preferably between 2 to 20 mM and EDTA is present at a concentration allowing the presence of free calcium ions in the composition.

6. A composition according to claim 5, which comprises EDTA at a concentration between 0.001 mM to 2 mM.

7. An aqueous composition comprising a therapeutically effective amount of Factor VIII, further characterized in that:
(i) the composition comprises calcium ions at a concentration between 0.5 to 30 mM;
(ii) the pH of the composition is adjusted to about 6.25;
(iii) the composition comprises a strong ligand at a concentration no higher than that of calcium ions; and
(iv) the composition is substantially free of other excipients which are free forms of medium-strength ligands or strong ligands.

8. A composition according to claim 7 wherein the strong ligand is EDTA.

9. A composition according to claim 1, which comprises at least 100 mM of sodium chloride or potassium chloride.

10. A composition according to claim 1, characterized in that it comprises benzoate ion and TRIS, wherein the TRIS and benzoate are present each at a concentration of between 1 to 100 mM.

11. A composition according to claim 1, which further comprises a surfactant, such as polysorbate 20, polysorbate 60, polysorbate 80, poloxamer 188 or poloxamer 407.

12. A composition according to claim 1, which further comprises a preservative approved for use in drug products.

13. A composition according to claim 12, wherein the preservative is selected from the group consisting of phenol, m-cresol, benzyl alcohol, propylparaben, benzalkonium chloride and benzethonium chloride.

14. A composition according to claim 1 which further comprises 1,2-propanediol at a concentration at least 100 mM.

15. The composition according to claim 1, wherein the Factor VIII is present in an amount between 50 and 1000 IU/ml.

16. An aqueous composition according to claim 1, which is stored in a sealed container under a headspace substantially free of carbon dioxide.

17. An aqueous composition comprising a therapeutically effective amount of Factor VIII, further characterized in that:
the composition comprises calcium ions at a concentration between 8 to 15 mM;
(ii) the composition comprises benzoate ion and TRIS, each at a concentration between 15 to 25 mM;
(iii) the composition comprises sodium chloride at a concentration between 300 to 500 mM;
(iv) the pH of the composition is adjusted to 6.25;
(v) the composition comprises EDTA at a concentration between 0.25 mM to 0.5 mM;
(vi) the composition comprises polysorbate 80 at a concentration between 10 to 50 mg/L or poloxamer 188 at a concentration between 0.2 to 3 mg/mL;
(vii) the composition is stored in a sealed container under a headspace substantially free from carbon dioxide; and
(viii) the composition is sterile.

18. A substantially water-free composition obtainable by removing water, from a composition according to claim 1.
